(19) United States
(12) Patent Application Publication
(10) Pub. No.: US 2019/0307336 A1
(43) Pub. Date: Oct. 10, 2019

(54) PULSE WAVE MEASUREMENT DEVICE, PULSE WAVE MEASUREMENT METHOD, AND BLOOD PRESSURE MEASUREMENT DEVICE

(71) Applicants: OMRON CORPORATION, Kyoto-shi (JP); OMRON HEALTHCARE CO., LTD., Muko-shi (JP)

(72) Inventors: Kenji FUJII, Kyoto (JP); Naoki MATSUMOTO, Kyoto (JP); Kentaro MORI, Kyoto (JP)

(73) Assignees: OMRON CORPORATION, Kyoto-shi (JP); OMRON HEALTHCARE CO., LTD., Muko-shi (JP)

(21) Appl. No.: 16/448,456

(22) Filed: Jun. 21, 2019

Related U.S. Application Data
(63) Continuation of application No. PCT/JP2017/038867, filed on Oct. 27, 2017.

(30) Foreign Application Priority Data
Dec. 28, 2016 (JP) 2016-254767

(51) Int. Cl. A61B 5/021; A61B 5/022; A61B 5/00

(52) U.S. Cl. CPC A61B 5/02125; A61B 5/6824; A61B 5/02225

(57) ABSTRACT

A pulse wave measurement device includes: a belt to be attached around a measurement site; first and second pulse wave sensors which are mounted on the belt in a state of being spaced apart from each other in a width direction of the belt and detect pulse waves at portions of an artery passing through the measurement site, the portions individually facing the first and second pulse wave sensors; and a pressing member capable of pressing the first and second pulse wave sensors against the measurement site while varying a pressing force. First and second pulse wave signals which the first and second pulse wave sensors output respectively in a time series are acquired, and a cross-correlation coefficient between waveforms of the first and second pulse wave signals is calculated.

### CROSS-REFERENCE TO RELATED APPLICATION

This is a continuation application of International Application No. PCT/JP2017/038867, with an International filing date of Oct. 27, 2017, which claim priority of Japanese Patent Application No. 2016-254767 filed on Dec. 28, 2016, the entire content of which is hereby incorporated by reference.

### TECHNICAL FIELD

This invention relates to a pulse wave measurement device and a pulse wave measurement method, and more specifically, relates to a pulse wave measurement device and a pulse wave measurement method which noninvasively measure a transit time of a pulse wave (a pulse transit time: PTT) that transits through an artery.

Moreover, this invention relates to a blood pressure measurement device that is provided with the pulse wave measurement device as described above and calculates a blood pressure using a corresponding equation between the pulse transit time and the blood pressure.

### BACKGROUND ART

Heretofore, for example, as disclosed in Patent Literature 1 (Japanese Unexamined Patent Application Publication No. H2-213324), there has been known a technology of fixing and arranging a small rubber bag and a medium rubber bag in a cloth bag (a cuff) in a state of being spaced apart from each other with respect to a width direction (corresponding to a longitudinal direction of the upper arm) of this cloth bag and measuring a time difference (a pulse transit time) between pulse wave signals individually detected by the small rubber bag and the medium rubber bag. In the cloth bag, a large rubber bag for measuring a blood pressure by an oscillometric method is disposed between the small rubber bag and the medium rubber bag.

### SUMMARY OF INVENTION

In Patent Literature 1, the pulse transit time is measured while performing pressurization/decompression operations for the small rubber bag and the medium rubber bag so that pressures in both the bag become the same as a pressure in the large rubber bag. That is, the pulse transit time is measured while changing the pressures in the small rubber bag and the medium rubber bag, in other words, while changing measurement conditions. Therefore, there is a problem that measurement accuracy for the pulse transit time is not good.

For example, assumed is a mode of mounting two pulse wave sensors on a belt (or a cuff) for attaching a wearable instrument on the wrist in a state where the two pulse wave sensors are spaced apart from each other with respect to a width direction (corresponding to a longitudinal direction of the wrist) of this belt and measuring a time difference (a pulse transit time) between pulse wave signals individually detected by the two pulse wave sensors. In this mode, a width of the belt is limited in order to reduce discomfort when the belt is attached, and hence, a distance between the two pulse wave sensors is limited to a relatively short length. Therefore, it is particularly required to improve the measurement accuracy for the pulse transit time.

Therefore, it is an object of this invention to provide a pulse wave measurement device and a pulse wave measurement method which are capable of improving the measurement accuracy for the pulse transit time.

It is another object of this invention is to provide a blood pressure measurement device that is provided with the pulse wave measurement device as described above and calculates a blood pressure using a corresponding equation between the pulse transit time and a blood pressure.

In order to solve the above-mentioned problem, a pulse wave measurement device of the present disclosure comprises:

a belt to be attached around a measurement site;

first and second pulse wave sensors which are mounted on the belt in a state of being spaced apart from each other in a width direction of the belt and detect pulse waves at portions of an artery passing through the measurement site, the portions individually facing the first and second pulse wave sensors;

a pressing member that is mounted on the belt and is capable of pressing the first and second pulse wave sensors against the measurement site while varying a pressing force;

a cross-correlation coefficient calculation unit that acquires first and second pulse wave signals which the first and second pulse wave sensors output respectively in a time series and calculates a cross-correlation coefficient between waveforms of the first and second pulse wave signals;

a search processing unit that varies and sets the pressing force generated by the pressing member and determines whether the cross-correlation coefficient which the cross-correlation coefficient calculation unit calculates exceeds a predetermined threshold value; and

a measurement processing unit that sets the pressing force generated by the pressing member to a value determined by the search processing unit, at which the cross-correlation coefficient exceeds the threshold value, and in a state of the pressing force's setting kept, acquires a time difference between the first and second pulse wave signals as a pulse transit time.

In this description, “measurement site” refers to a region through which an artery passes. For example, the measurement site may be an arm such as a wrist and an upper arm, or may be a leg such as an ankle and a thigh.

Moreover, “belt” refers to a member of band-like shape attached around the measurement site, regardless of its name. For example, the name of the belt may be “band”, “cuff” or the like in place of the belt.

Moreover, “width direction” of the belt corresponds to a longitudinal direction of the measurement site.

Furthermore, “cross-correlation coefficient” means a sample correlation coefficient (also referred to as Pearson product-moment correlation coefficient). For example, when data string {xᵢ} and a data string {yᵢ} which are composed of two sets of numeric values (where i=1, 2 . . . , n) are given, a cross-correlation coefficient r between the data string {xᵢ} and the data string {yᵢ} is defined by Equation (Eq. 1) illustrated in FIG. 11. Herein, x and y added with top bars in Equation (Eq. 1) represent average values of x and y, respectively.

In another aspect, a blood pressure measurement device of the present disclosure comprises:

the above-described pulse wave measurement device; and

a first blood pressure calculation unit that calculates a blood pressure based on the pulse transit time, which is acquired by the measurement processing unit, using a predetermined corresponding equation between the pulse transit time and the blood pressure.

In another aspect, a pulse wave measurement method of the present disclosure is a pulse wave measurement method of measuring a pulse wave of a measurement site by including:

a belt to be attached around the measurement site;

first and second pulse wave sensors mounted on the belt in a state of being spaced apart from each other with in a width direction of the belt; and

a pressing member that is mounted on the belt and is capable of pressing the first and second pulse wave sensors against the measurement site while varying a pressing force, the pulse wave measurement method comprising:

detecting pulse waves at portions of an artery passing through the measurement site by the first and the second pulse wave sensors in a state where the belt is attached around the measurement site and the pressing member presses the first and second pulse wave sensors against the measurement site by a certain pressing force, the portions individually facing the first and second pulse wave sensors;

acquiring first and second pulse wave signals which the first and second pulse wave sensors output respectively in a time series and calculating a cross-correlation coefficient between waveforms of the pulse wave signals;

varying and setting the pressing force generated by the pressing member and determining whether the cross-correlation coefficient exceeds a predetermined threshold value regarding the pressing force; and

setting the pressing force generated by the pressing member to a value determined by the search processing unit, at which the cross-correlation coefficient exceeds the threshold value, and in a state of the pressing force's setting kept, acquiring a time difference between the first and second pulse wave signals as a pulse transit time.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a perspective view illustrating an exterior appearance of a wrist sphygmomanometer of an embodiment according to a blood pressure measurement device provided with a pulse wave measurement device of this invention.

FIG. 2 is a view schematically illustrating a cross section perpendicular to a longitudinal direction of a wrist in a state where the sphygmomanometer is attached to a left wrist as the wrist.

FIG. 3 is a view illustrating a planar layout of impedance measuring electrodes which constitute first and second pulse wave sensors in the state where the sphygmomanometer is attached to the left wrist.

FIG. 4 is a diagram illustrating a block configuration of a control system of the sphygmomanometer.

FIG. 5A is a view schematically illustrating a cross section along a longitudinal direction of the wrist in the state where the sphygmomanometer is attached to the left wrist.

FIG. 5B is a view illustrating waveforms of first and second pulse wave signals outputted by the first and second pulse wave sensors, respectively.

FIG. 6 is a diagram illustrating an operation flow at a time when the sphygmomanometer performs blood pressure measurement by an oscillometric method.

FIG. 7 is a diagram illustrating changes of a cuff pressure and a pulse wave signal, the changes being caused by the operation flow of FIG. 6.

FIG. 8 is a diagram illustrating an operation flow at a time when the sphygmomanometer executes a pulse wave measurement method of the embodiment, acquires a pulse transit time (PTT), and performs blood pressure measurement (estimation) based on the pulse transit time.

FIG. 9 is a diagram illustrating a relationship between a pressing force against the impedance measuring electrode and a cross-correlation coefficient between waveforms of first and second pulse wave signals outputted from the first and second pulse wave sensors, respectively.

FIG. 10A is a scatter diagram illustrating a relationship between pulse transit time (PTT) acquired under a condition where a pressing force (a cuff pressure) is set to 40 mmHg by the sphygmomanometer for a variety of users (subjects) and a systolic blood pressure (SBP) obtained by the blood pressure measurement by the oscillometric method for the variety of users.

FIG. 10B is a scatter diagram illustrating a relationship between pulse transit time (PTT) acquired under a condition where the pressing force (the cuff pressure) is set to 130 mmHg by the sphygmomanometer for the variety of users and the systolic blood pressure (SBP) obtained by the blood pressure measurement by the oscillometric method for the variety of users.

FIG. 11 is a diagram exemplifying an equation representing a cross-correlation coefficient r between a data string {xᵢ} and a data string {yᵢ}.

FIG. 12 is a diagram illustrating an example of a predetermined corresponding equation between a pulse transit time and a blood pressure.

FIG. 13 is a diagram illustrating another example of the predetermined corresponding equation between the pulse transit time and the blood pressure.

FIG. 14 is a diagram illustrating still another example of the predetermined corresponding equation between the pulse transit time and the blood pressure.

### DESCRIPTION OF EMBODIMENT

Hereinafter, a detailed description will be given of embodiments of this invention with reference to the drawings.

(Configuration of Sphygmomanometer)

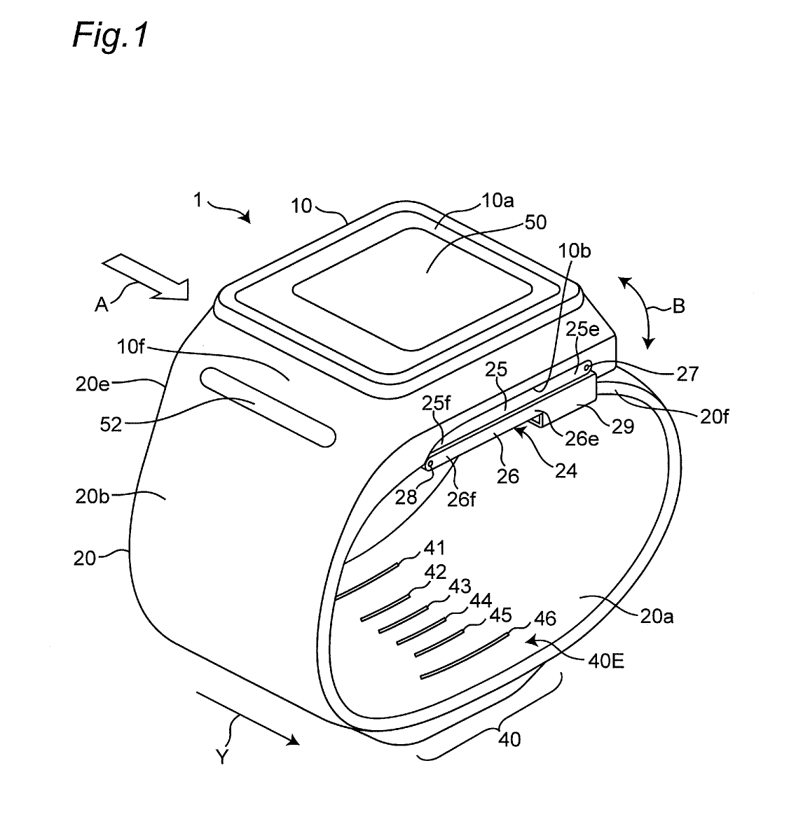

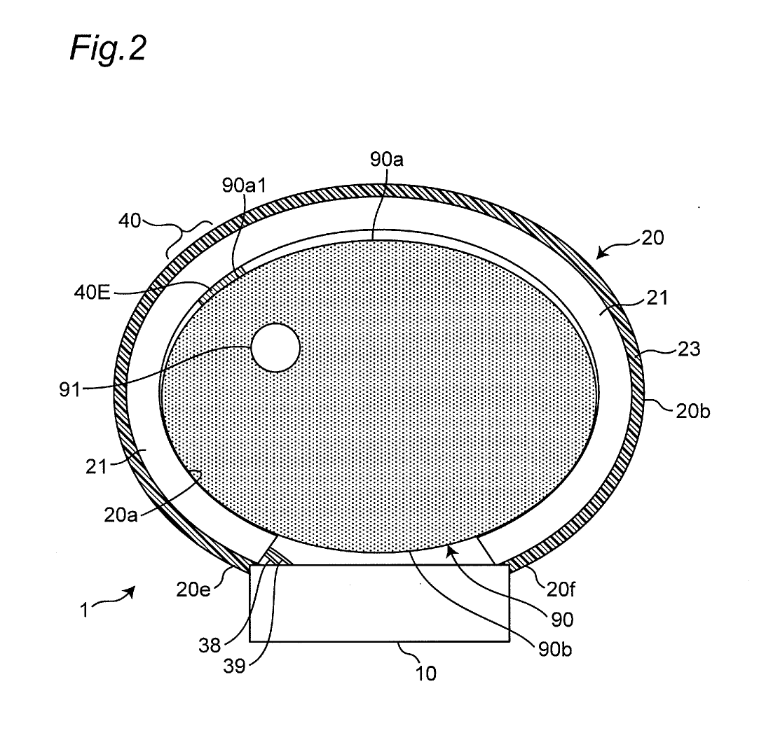

FIG. 1 illustrates a perspective view of an exterior appearance of a wrist sphygmomanometer (of which entirety is denoted by reference numeral 1) of an embodiment according to a blood pressure measurement device provided with a pulse wave measurement device of this invention. Moreover, FIG. 2 schematically illustrates a cross section of the sphygmomanometer 1, which is perpendicular to a longitudinal direction of a left wrist 90 as a measurement site, in a state where the sphygmomanometer 1 is attached to the left wrist 90 (hereinafter, this state will be referred to as “attached state”).

As illustrated in these drawings, this sphygmomanometer 1 roughly includes: a belt 20 to be attached around the left wrist 90 of a user; and a body 10 integrally attached to this belt 20.

As well seen from FIG. 1, the belt 20 has an inner circumferential surface 20a that has an elongated band-like shape so as to surround the left wrist 90 along a circumferential direction of the left wrist and is to be brought into contact with the left wrist 90; and an outer circumferential surface 20b opposite with this inner circumferential surface 20a. A dimension (width dimension) in a width direction Y of the belt 20 is set to approximately 30 mm in this example.

The body 10 is integrally provided on one end portion 20e of the belt 20 with respect to the circumferential direction by, in this example, integral molding. Note that the belt 20 and the body 10 may be formed separately from each other, and the body 10 may be integrally attached to the belt 20 with an engagement member (for example, a hinge or the like) interposed therebetween. In this example, a region in which the body 10 is disposed is scheduled to correspond to a backside surface (back-of-hand-side surface) 90b of the left wrist 90 in the attached state (see FIG. 2). In FIG. 2, a radial artery 91 that passes through a vicinity of a palm-side surface 90a in the left wrist 90 is illustrated.

As well seen from FIG. 1, the body 10 has a three-dimensional shape having a thickness in a direction perpendicular to the outer circumferential surface 20b of the belt 20. This body 10 is formed compact and thin so as not to disturb daily activities of the user. In this example, the body 10 has an outline of a truncated quadrangular pyramid shape protruding outward from the belt 20.

A display 50 that forms a display screen is provided on a top surface (farthest surface from the measurement site) 10a of the body 10. Moreover, an operation portion 52 for inputting an instruction from the user is provided along a side surface (side surface on a left front side in FIG. 1) 10f of the body 10.

In the belt 20, on a region between the one end portion 20e and the other end portion 20f with respect to the circumferential direction, an impedance measurement portion 40 that constitutes first and second pulse wave sensors is provided. In the belt 20, on the inner circumferential surface 20a in a region in which the impedance measurement portion 40 is disposed, there are arranged six plate-shaped (or sheet-shaped) electrodes 41 to 46 spaced apart from one another with respect to the width direction Y of the belt 20. The entirety of the electrodes 41 to 46 is referred to as “electrode group”, which is denoted by reference numeral 40E, and will be described later in detail. In this example, a region in which the electrode group 40E is disposed is to correspond to the radial artery 91 of the left wrist 90 in the attached state (see FIG. 2).

As illustrated in FIG. 1, a bottom surface (a surface closest to the measurement site) 10b of the body 10 and the end portion 20f of the belt 20 are connected to each other by a three-fold buckle 24. This buckle 24 includes: a first plate-shaped member 25 disposed on an outer circumference side and a second plate-shaped member 26 disposed on an inner circumference side. The one end portion 25e of the first plate-shaped member 25 is rotatably attached to the body 10 while interposing therebetween a coupling bar 27 extending along the width direction Y. The other end portion 25f of the first plate-shaped member 25 is rotatably attached to one end portion 26e of the second plate-shaped member 26 while interposing therebetween a coupling bar 28 extending along the width direction Y. The other end portion 26f of the second plate-shaped member 26 is fixed to a vicinity of the end portion 20f of the belt 20 by a fixing portion 29. Note that an attached position of the fixing portion 29 with respect to the circumferential direction of the belt 20 is set variable in advance in matching with a circumferential length of the left wrist 90 of the user. In this way, this sphygmomanometer 1 (the belt 20) is composed into a substantially annular shape as a whole, and the bottom surface 10b of the body 10 and the end portion 20f of the belt 20 are made openable by the buckle 24 in a direction of arrow B.

At the time of attaching this sphygmomanometer 1 to the left wrist 90, in a state where the buckle 24 is opened to increase a diameter of a ring of the belt 20, the user puts the left hand through the belt 20 in an orientation indicated by arrow A in FIG. 1. Then, as illustrated in FIG. 2, the user adjusts an angular position of the belt 20 around the left wrist 90, and locates the impedance measurement portion 40 of the belt 20 above the radial artery 91 passing through the left wrist 90. In this way, the electrode group 40E of the impedance measurement portion 40 abuts against a portion 90a1 corresponding to the radial artery 91 in the palm-side surface 90a of the left wrist 90. In this state, the user closes and fixes the buckle 24. In this way, the user attaches the sphygmomanometer 1 (the belt 20) to the left wrist 90.

As illustrated in FIG. 2, in this example, the belt 20 includes: a band-shaped body 23 that forms the outer circumferential surface 20b: and a pressing cuff 21 as a pressing member attached along an inner circumferential surface of this band-shaped body 23. In this example, the band-shaped body 23 is made of a plastic material that has flexibility with respect to a thickness direction thereof and is substantially non-stretchable with respect to a circumferential direction (a longitudinal direction) thereof. In this example, the pressing cuff 21 is composed as a fluid bag formed in such a manner that two stretchable polyurethane sheets are caused to face each other in a thickness direction thereof and peripheral edge portions of the polyurethane sheets are welded to each other. In the inner circumferential surface 20a of the pressing cuff 21 (the belt 20), on a region thereof corresponding to the radial artery 91 of the left wrist 90, the electrode group 40E of the impedance measurement portion 40 is disposed as already mentioned.

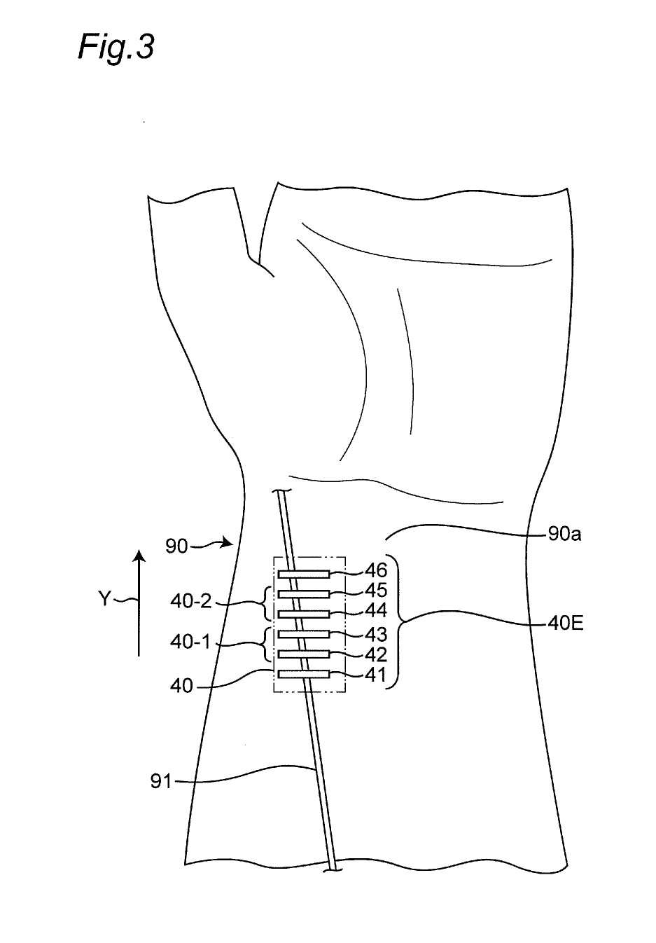

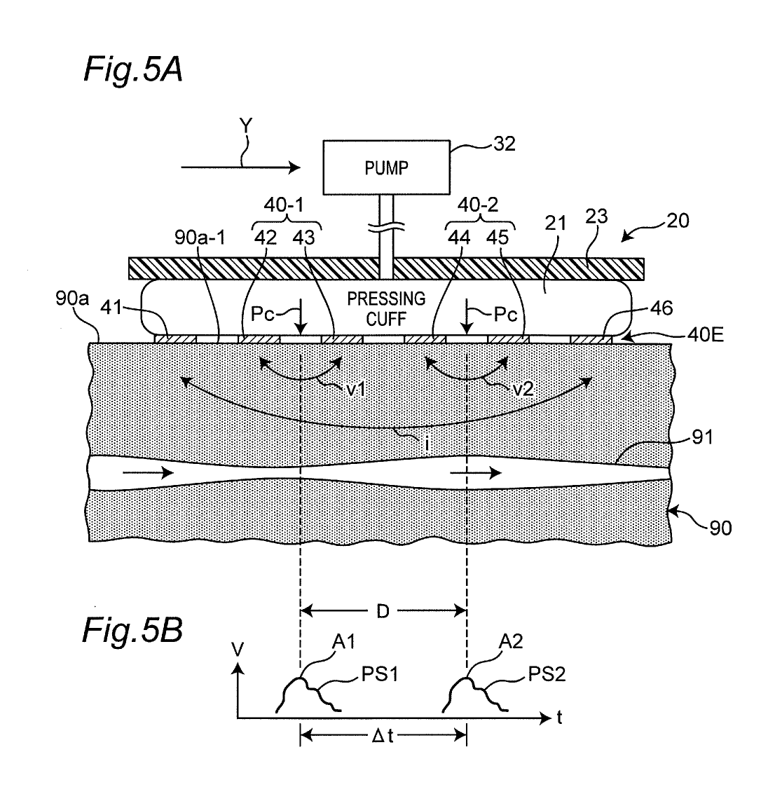

As illustrated in FIG. 3, in the attached state, the electrode group 40E of the impedance measurement portion 40 is arrayed along a longitudinal direction of the wrist (which is equivalent to the width direction Y of the belt 20) so as to correspond to the radial artery 91 of the left wrist 90. The electrode group 40E includes: a pair of current electrodes 41 and 46 for energization, which are disposed on both sides in the width direction Y; a first pair of detection electrodes 42 and 43 for voltage detection, which are disposed between the pair of current electrodes 41 and 46 and forms a first pulse wave sensor 40-1; and a second pair of detection electrodes 44 and 45 for voltage detection, which are disposed between the pair of current electrodes 41 and 46 and forms a second pulse wave sensor 40-2. The second pair of detection electrodes 44 and 45 are disposed so as to correspond to a downstream portion of the first pair of detection electrodes 42 and 43 in a bloodstream of the radial artery 91. With respect to the width direction Y, a distance D (see FIG. 5A) between a center of the first pair of detection electrodes 42 and 43 and a center of the second pair of detection electrodes 44 and 45 is set to 20 mm in this example. This distance D corresponds to a substantial interval between the first pulse wave sensor 40-1 and the second pulse wave sensor 40-2. Moreover, with respect to the width direction Y, each of an interval between the first pair of detection electrodes 42 and 43 and an interval between the second pair of detection electrodes 44 and 45 is set to 2 mm in this example.

The electrode group 40E as described above can be composed to be flat. Hence, in this sphygmomanometer 1, the belt 20 can be composed to be thin as a whole.

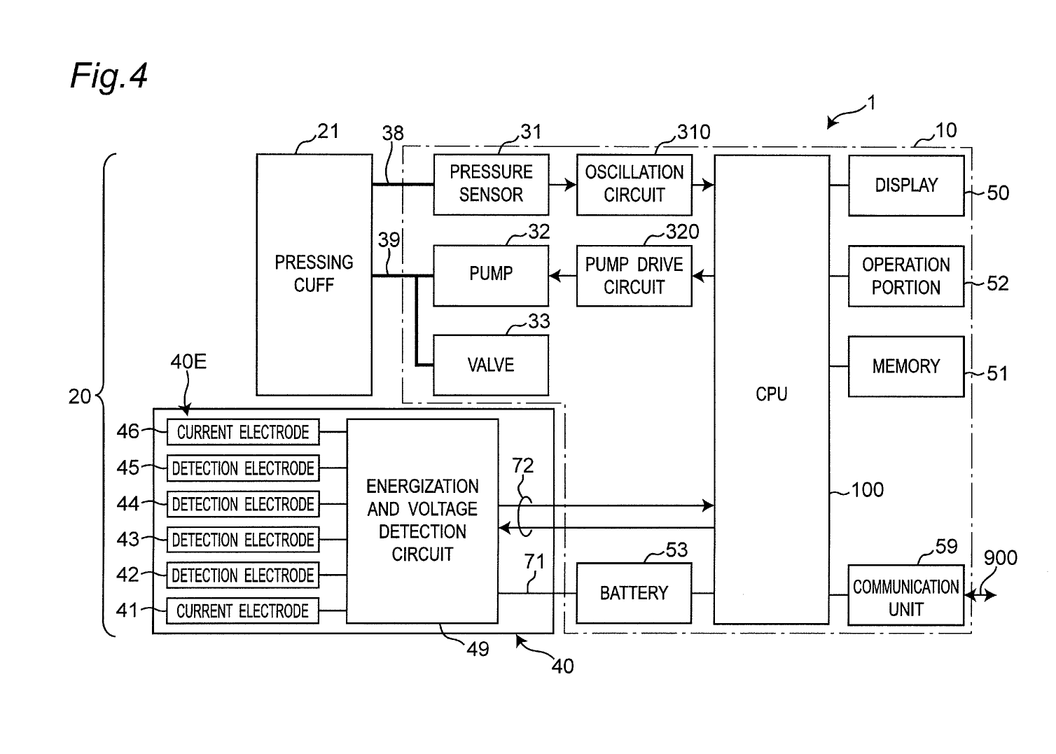

FIG. 4 illustrates a block configuration of a control system of the sphygmomanometer 1. On the body 10 of the sphygmomanometer 1, in addition to the already-described display 50 and operation portion 52, there are mounted: a central processing unit (CPU) 100 as a control unit; a memory 51 as a storage unit; a communication unit 59; a pressure sensor 31; a pump 32; a valve 33; an oscillation circuit 310 that converts, into a frequency, an output sent from the pressure sensor 31; a pump drive circuit 320 that drives the pump 32. Moreover, an energization and voltage detection circuit 49 is mounted on the impedance measurement portion 40 in addition to the electrode group 40E already described.

In this example, the display 50 is composed of an organic electro luminescence (EL) display, and displays information regarding blood pressure measurement, such as a blood pressure measurement result, and other information in accordance with a control signal sent from the CPU 100. Note that the display 50 is not limited to the organic EL display, and for example, may be composed of a display of another type, such as a liquid crystal display (LCD).

In this example, the operation portion 52 is composed of a push-type switch, and inputs, to the CPU 100, an operation signal corresponding to a user's instruction to start or stop the measurement of the blood pressure. Note that the operation portion 52 is not limited to the push-type switch, and for example, may be a touch panel-type switch of a pressure sensitive type (a resistance type) or a proximity type (electrostatic capacitance-type). Moreover, the operation portion 52 may be provided with a microphone (not illustrated), and may input the instruction to start the measurement of the blood pressure by user's voice.

The memory 51 non-transitorily stores data of a program for controlling the sphygmomanometer 1, data for use in controlling the sphygmomanometer 1, setting data for setting a variety of functions of the sphygmomanometer 1, data of a measurement result of a blood pressure value, and the like. Moreover, the memory 51 is used as a work memory when the program is executed.

As a control unit, the CPU 100 executes the variety of functions in accordance with the program for controlling the sphygmomanometer 1, the program being stored in the memory 51. For example, in the case of executing blood pressure measurement by the oscillometric method, the CPU 100 performs control to drive the pump 32 (and the valve 33) in response to the instruction to start the blood pressure measurement, which is issued from the operation portion 52, on the basis of a signal sent from the pressure sensor 31. Moreover, in this example, the CPU 100 performs control to calculate the blood pressure value on the basis of a signal sent from the pressure sensor 31.

The communication unit 59 is controlled by the CPU 100 to transmit predetermined information to an external device via a network 900, and to receive, via the network 900, information sent from the external device and transfer the received information to the CPU 100. This communication via the network 900 may be either wireless communication or wired communication. In this embodiment, the network 900 is the Internet; however, is not limited to this, and may be another type of network such as a local area network (LAN) in a hospital, or may be one-to-one communication using a USB cable. This communication unit 59 may include a micro USB connector.

The pump 32 and the valve 33 are connected to the pressing cuff 21 via an air pipe 39, and the pressure sensor 31 is connected to the pressing cuff 21 via an air pipe 38. Note that the air pipes 39 and 38 may be a common single pipe. Via the air pipe 38, the pressure sensor 31 detects a pressure in the pressing cuff 21. In this example, the pump 32 is composed of a piezoelectric pump, and in order to increase a pressure (a cuff pressure) in the pressing cuff 21, supplies air as a fluid for pressurization to the pressing cuff 21 through the air pipe 39. The valve 33 is mounted on the pump 32, and is configured to be subjected to opening/closing control following on/off of the pump 32. That is, when the pump 32 is turned on, the valve 33 is closed to enclose air in the pressing cuff 21, and meanwhile, when the pump 32 is turned off, the valve 33 is opened to discharge the air in the pressing cuff 21 to the atmosphere through the air pipe 39. Note that the valve 33 has a function of a check valve, and does not allow the discharged air to flow backward. The pump drive circuit 320 drives the pump 32 on the basis of the control signal given from the CPU 100.

In this example, the pressure sensor 31 is a piezo-resistance-type pressure sensor, and through the air pipe 38, detects a pressure of the belt 20 (the pressing cuff 21), in this example, a pressure taking the atmospheric pressure as a reference (zero), and outputs the detected pressure as a time-series signal. The oscillation circuit 310 oscillates on the basis of an electrical signal value based on a change of an electrical resistance, the change being caused by a piezo-resistance effect sent from the pressure sensor 31, and outputs, to the CPU 100, a frequency signal having a frequency corresponding to the electrical signal value of the pressure sensor 31. In this example, the output of the pressure sensor 31 is used for controlling the pressure of the pressing cuff 21 and calculating the blood pressure value (including a systolic blood pressure (SBP) and a diastolic blood pressure DBP) by the oscillometric method.

A battery 53 supplies electrical power to elements mounted on the body 10, in this example, to the respective elements which are the CPU 100, the pressure sensor 31, the pump 32, the valve 33, the display 50, the memory 51, the communication unit 59, the oscillation circuit 310, and the pump drive circuit 320. Moreover, the battery 53 also supplies electrical power to the energization and voltage detection circuit 49 of the impedance measurement portion 40 through a wire 71. This wire 71 is provided to extend along the circumferential direction of the belt 20 between the body 10 and the impedance measurement portion 40 in a state of being sandwiched between the band-shaped body 23 of the belt 20 and the pressing cuff 21 together with wires 72 for a signal.

The energization and voltage detection circuit 49 of the impedance measurement portion 40 is controlled by the CPU 100, and at an operation time thereof, as illustrated in FIG. 5A, flows a high-frequency constant current i, in this example, with a frequency of 50 kHz and a current value of 1 mA between the pair of current electrodes 41 and 46 disposed on both sides with respect to the longitudinal direction (equivalent to the width direction Y of the belt 20) of the wrist. In this state, the energization and voltage detection circuit 49 detects a voltage signal v1 between the first pair of detection electrodes 42 and 43 which form the first pulse wave sensor 40-1 and a voltage signal v2 between the second pair of detection electrodes 44 and 45 which form the second pulse wave sensor 40-2. These voltage signals v1 and v2 represent changes of electrical impedances, which are caused by a pulse wave of a blood flow in the radial artery 91 in portions which the first pulse wave sensor 40-1 and the second pulse wave sensor 40-2 individually face, the portions belonging to the palm-side surface 90a of the left wrist 90 (impedance system). The energization and voltage detection circuit 49 rectify, amplify and filtrate these voltage signals v1 and v2, and output a first pulse wave signal PS1 and a second pulse wave signal PS2, which have a mount-shaped waveform as illustrated in FIG. 5B, in a time series. In this example, the voltage signals v1 and v2 are approximately 1 mV. Moreover, peaks A1 and A2 of the first pulse wave signal PS1 and the second pulse wave signal PS2 are approximately 1 v in this example.

Note that, if a pulse wave velocity (PWV) of the blood flow of the radial artery 91 is within a range of 1000 cm/s to 2000 cm/s, a time difference Δt between the first pulse wave signal PS1 and the second pulse wave signal PS2 stays within a range of 1.0 ms to 2.0 ms since the substantial interval D between the first pulse wave sensor 40-1 and the second pulse wave sensor 40-2 is 20 mm (D=20 mm).

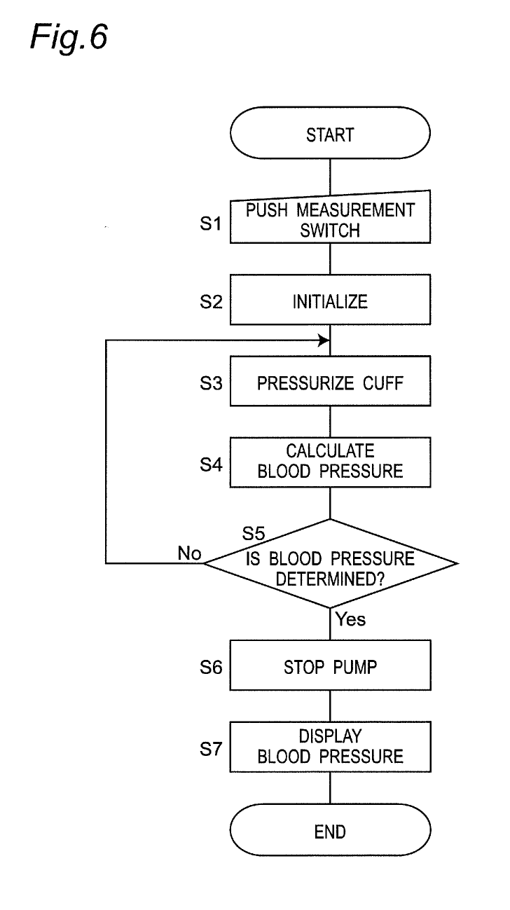

(Operation of blood pressure measurement by oscillometric method) FIG. 6 illustrates an operation flow at a time when the sphygmomanometer 1 performs the blood pressure measurement by the oscillometric method.

When the user issues an instruction to perform the blood pressure measurement by the oscillometric method using the push-type switch as the operation portion 52 provided in the body 10 (Step S1), the CPU 100 starts an operation, and initializes a memory area for processing (Step S2). Moreover, the CPU 100 turns off the pump 32 via the pump drive circuit 320, opens the valve 33, and exhausts the air in the pressing cuff 21. Subsequently, the CPU 100 performs control to set an output value of the pressure sensor 31 at the present point of time as a value corresponding to the atmospheric pressure (0 mmHg adjustment).

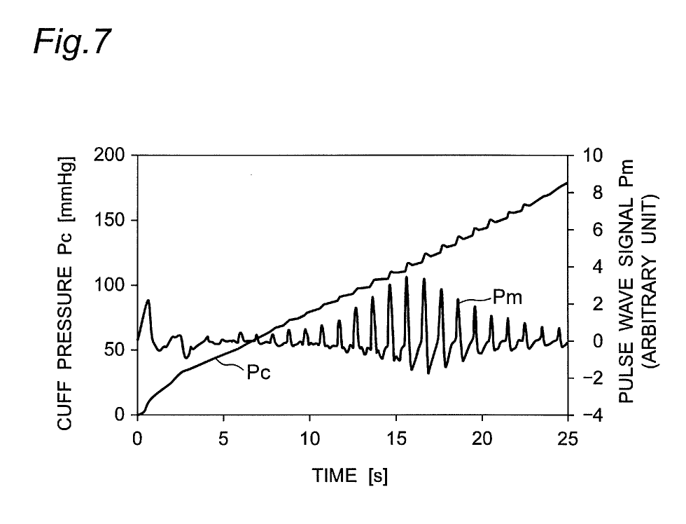

Subsequently, the CPU 100 works as a pressure control unit, closes the valve 33, and thereafter, drives the pump 32 via the pump drive circuit 320, and performs control to send air to the pressing cuff 21. In this way, the CPU 100 inflates the pressing cuff 21, and gradually increases the cuff pressure Pc (see FIG. 7) (Step S3 in FIG. 6).

In this pressurization process, the CPU 100 monitors the cuff pressure Pc by the pressure sensor 31 in order to calculate the blood pressure value, and acquires, as a pulse wave signal Pm as illustrated in FIG. 7, a variable component of an artery volume, which occurs in the radial artery 91 of the left wrist 90 as the measurement site.

Next, in Step S4 in FIG. 6, the CPU 100 works as a second blood pressure calculation unit, and on the basis of the pulse wave signal Pm acquired at this point of time, attempts to calculate the blood pressure value (the systolic blood pressure SBP and the diastolic blood pressure DBP) by applying a known algorithm by the oscillometric method.

When the blood pressure value cannot be calculated yet at this point of time due to a lack of data (NO in Step S5), the CPU 100 repeats processing of Steps S3 to S5 as long as the cuff pressure Pc does not reach an upper limit pressure (that is predetermined, for example, at 300 mmHg for safety).

When the blood pressure value can be calculated as described above (YES in Step S5), the CPU 100 stops the pump 32, opens the valve 33, and performs control to exhaust the air in the pressing cuff 21 (Step S6). And finally, the CPU 100 displays the measurement result of the blood pressure value on the display 50, and records the measurement result in the memory 51 (Step S7).

Note that the calculation of the blood pressure value may be performed not only in the pressurization process but also in a decompression process.

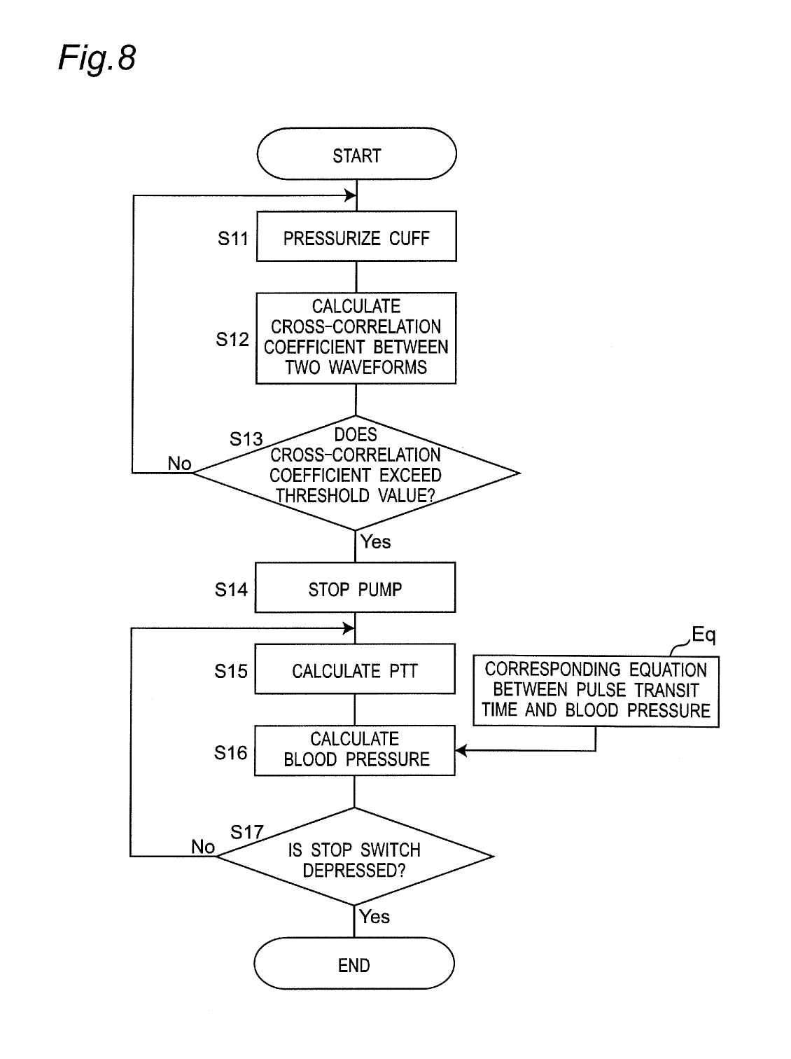

(Operation of blood pressure measurement based on pulse transit time) FIG. 8 illustrates an operation flow at a time when the sphygmomanometer 1 executes a pulse wave measurement method of the embodiment, acquires the pulse transit time (PTT), and performs the blood pressure measurement (estimation) based on the pulse transit time.

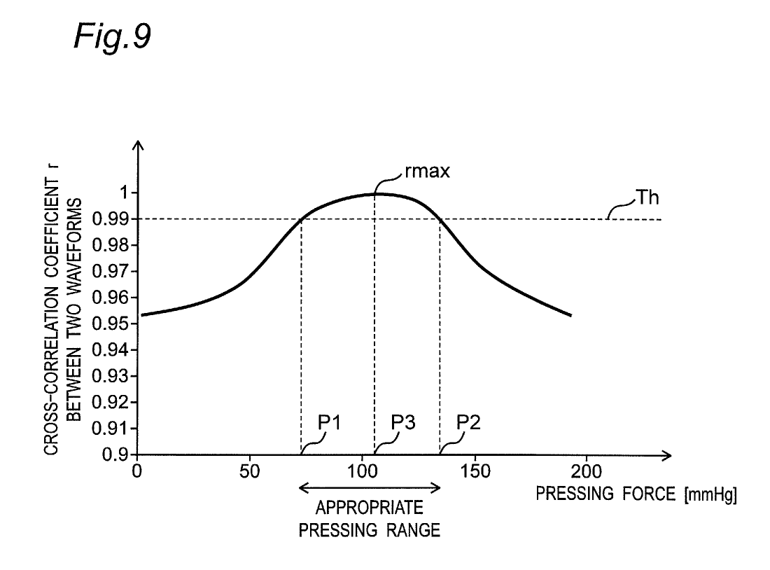

This operation flow was created on the basis of a result of an experiment performed by the inventors of the present invention. That is, in accordance with the experiment performed by the inventors of the present invention, it was found that, when the pressing forces (which are equal to the cuff pressure Pc by the pressing cuff 21) against the left wrist 90 as the measurement site at the first pulse wave sensor 40-1 (including the first pair of detection electrodes 42 and 43) and the second pulse wave sensor 40-2 (including the second pair of detection electrodes 44 and 45) were gradually increased from zero as illustrated in FIG. 9, then the cross-correlation coefficient r between the pulse wave signals PS1 and PS2 was gradually increased following the increase of the pressing forces, exhibited a maximum value max, and then was gradually decreased. This operation flow is based on the thought that a range where the cross-correlation coefficient r exceeds a predetermined threshold value Th (Th=0.99 in this example) is an appropriate range of the pressing force (this is referred to as “appropriate pressing range”). In this example, the appropriate pressing range is a range where the pressing force (the cuff pressure Pc) is from a lower limit value P1≈72 mmHg to an upper limit value P2≈135 mmHg.

When the user issues an instruction to perform the blood pressure measurement based on the PTT using the push-type switch as the operation portion 52 provided in the body 10 (Step S11 in FIG. 8), the CPU 100 starts an operation. That is, the CPU 100 works as a search processing unit, closes the valve 33, and in addition, drives the pump 32 via the pump drive circuit 320, and performs control to send air to the pressing cuff 21. In this way, the CPU 100 inflates the pressing cuff 21, and gradually increases the cuff pressure Pc (see FIG. 5A). In this example, the CPU 100 continuously increases the cuff pressure Pc at a constant speed (=5 mmHg/s). Note that the CPU 100 may increase the cuff pressure Pc step by step so that a time for calculating the cross-correlation coefficient r mentioned next can be ensured with ease.

In this pressurization process, the CPU 100 works as a cross-correlation coefficient calculation unit, acquires the first and second pulse wave signals PS1 and PS2 which the first pulse wave sensor 40-1 and the second pulse wave sensor 40-2 output respectively in a time series, and calculates the cross-correlation coefficient r between the waveforms of the first and second pulse wave signals PS1 and PS2 in real time (Step S12 of FIG. 8).

In addition to the above, the CPU 100 works as the search processing unit, and determines whether the calculated cross-correlation coefficient r exceeds the predetermined threshold value Th (=0.99) (Step S13 of FIG. 8). Herein, if the cross-correlation coefficient r is equal to or less than the threshold value Th (NO in Step S13 of FIG. 8), then the CPU 100 repeats processing of Step S11 to S13 until the cross-correlation coefficient r exceeds the threshold value Th. Then, when the cross-correlation coefficient r exceeds the threshold value Th (YES in Step S13 of FIG. 8), the CPU 100 stops the pump 32 (Step S14 of FIG. 8), and sets the cuff pressure Pc to a value at that point of time, that is, at the point of time when the cross-correlation coefficient r exceeds the threshold value Th. In this example, the cuff pressure Pc is set to the value at the point of time when the cross-correlation coefficient r exceeds the threshold value Th, that is, to P1 (≈72 mmHg) illustrated in FIG. 9.

In this state, the CPU 100 works as a measurement processing unit, and acquires, as the pulse transit time (PTT), the time difference Δt (see FIG. 5B) between the first and second pulse wave signals PS1 and PS2 (Step S15 of FIG. 8). More specifically, in this example, the CPU 100 acquires, as the pulse transit time (PTT), the time difference Δt between the peak A1 of the first pulse wave signal PS1 and the peak A2 of the second pulse wave signal PS2.

In the case of adopting such a procedure, the measurement accuracy for the pulse transit time can be improved. Moreover, since the cuff pressure Pc is set to the value at the point of time when the cross-correlation coefficient r exceeds the threshold value Th, the pulse transit time can be acquired without unnecessarily increasing the cuff pressure Pc. In this way, a physical load on the user can be reduced.

Next, the CPU 100 works as a first blood pressure calculation unit, and calculates (estimates) a blood pressure on the basis of the pulse transit time (PTT), which is acquired in Step S15, using a predetermined corresponding equation Eq between the pulse transit time and the blood pressure (Step S16 of FIG. 8). Herein, when the pulse transit time is represented as DT, and the blood pressure is represented as EBP, then the predetermined corresponding equation Eq between the pulse transit time and the blood pressure is provided as a known fractional function including a term of 1/DT², for example, as illustrated in Equation (Eq. 2) of FIG. 12 (for example, refer to Japanese Unexamined Patent Application Publication No. H10-201724). In Equation (Eq. 2), each of α and β represents a known coefficient or constant.

In the case of calculating (estimating) the blood pressure as described above, the measurement accuracy for the pulse transit time is improved as already described, and accordingly, the measurement accuracy for the blood pressure can be improved. Note that the measurement result of the blood pressure value is displayed on the display 50, and is recorded in the memory 51.

In this example, if the instruction to stop the measurement is not issued by the push-type switch as the operation portion 52 in Step S17 of FIG. 8 (NO in Step S17 of FIG. 8), the CPU 100 periodically repeats the calculation of the pulse transit time (PTT) (Step S15 of FIG. 8) and the calculation (estimation) of the blood pressure (Step S16 of FIG. 8) every time when the first and second pulse wave signals PS1 and PS2 are inputted in response to the pulse wave. The CPU 100 updates and displays the measurement result of the blood pressure value on the display 50, and stores and records the measurement result in the memory 51. Then, when the instruction to stop the measurement is issued in Step S17 of FIG. 8 (YES in Step S17 of FIG. 8), the CPU 100 ends the measurement operation.

In accordance with this sphygmomanometer 1, the blood pressure can be continuously measured during a long period by this blood pressure measurement based on the pulse transit time (PTT) in a state where the physical load on the user is light.

Moreover, in accordance with this sphygmomanometer 1, the blood pressure measurement (estimation) based on the pulse transit time and the blood pressure measurement by the oscillometric method can be performed by an integrated device. Hence, convenience for the user can be improved.

(Verification of Effect by Setting Pressing Force)

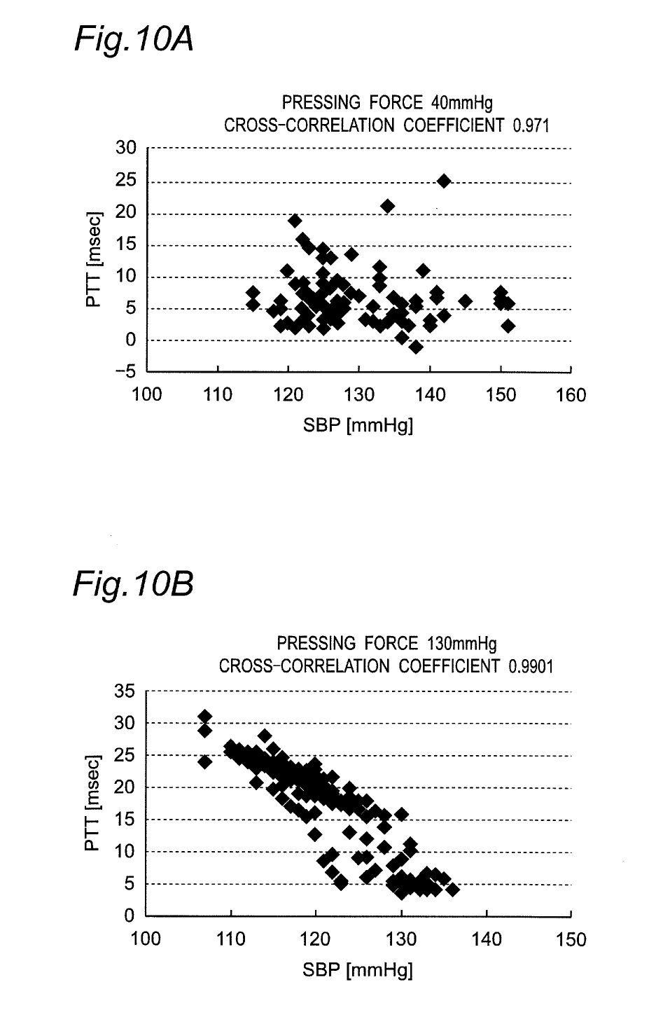

A scatter diagram of FIG. 10A illustrates a relationship between pulse transit time (PTT) acquired under a condition where the pressing force (the cuff pressure Pc) is set to 40 mmHg (that is less than the lower limit value P1 illustrated in FIG. 9) by the sphygmomanometer 1 for a variety of users (subjects) and the systolic blood pressure (SBP) obtained by the blood pressure measurement (Step S5 in FIG. 6) by the oscillometric method for the variety of users. The cross-correlation coefficient r between the waveforms of the first and second pulse wave signals PS1 and PS2 under such a condition where the pressing force is set was equal to 0.971 (r=0.971), which fell down below the threshold value Th (=0.99). As seen from FIG. 10A, there is hardly a correlation between the pulse transit time (PTT) and the systolic blood pressure (SBP). When the correlation coefficient was calculated by performing fitting using Equation (Eq. 2) of FIG. 12, the correlation coefficient was −0.07.

In contrast, a scatter diagram of FIG. 10B illustrates a relationship between pulse transit time (PTT) acquired under a condition where the pressing force (the cuff pressure Pc) is set to 130 mmHg (that is within the appropriate pressing range between the lower limit value P1 and the upper limit value P2 which are illustrated in FIG. 9) by the sphygmomanometer 1 for the above-mentioned variety of users and the systolic blood pressure (SBP) obtained by the blood pressure measurement (Step S5 in FIG. 6) by the oscillometric method for the variety of users. The cross-correlation coefficient r between the waveforms of the first and second pulse wave signals PS1 and PS2 under such a condition where the pressing force is set was equal to 0.9901 (r=0.9901), which exceeded the threshold value Th (=0.99). As seen from FIG. 10B, the correlation between the pulse transit time (PTT) and the systolic blood pressure (SBP) is strong. When the correlation coefficient was calculated by performing fitting using Equation (Eq. 2) of FIG. 12, the correlation coefficient was −0.90.

From these results of FIG. 10A and FIG. 10B, it could be verified that the correlation between the pulse transit time (PTT) and the systolic blood pressure (SBP) could be improved by setting the pressing force (the cuff pressure Pc) to the value at which the cross-correlation coefficient r exceeded the threshold value Th (=0.99) and acquiring the pulse transit time (PTT). A reason why the correlation between the pulse transit time (PTT) and the systolic blood pressure (SBP) could be improved as described above is conceived to be that the measurement accuracy for the pulse transit time (PTT) was improved by the setting of the pressing force according to the present invention. In this way, the measurement accuracy for the blood pressure can be improved.

### Modified Example

In the above example, in Steps S13 and S14 of FIG. 8, the pressing force (the cuff pressure Pc) was set to the value (the lower limit value P1 of the appropriate pressing range illustrated in FIG. 9) at the point of time when the cross-correlation coefficient r between the waveforms of the first and second pulse wave signals PS1 and PS2 exceeded the threshold value Th. However, the present invention is not limited to this. The CPU 100 may further perform the search, and may set the pressing force (the cuff pressure Pc) to a value (P3 illustrated in FIG. 9) at which the cross-correlation coefficient r exhibits the maximum value max. In the example of FIG. 9, this value P3 is almost equal to 106 mmHg (P3≈106 mmHg). In this way, the measurement accuracy for the pulse transit time can be further improved.

Moreover, in the above example, in Step S16 of FIG. 8, Equation (Eq. 2) of FIG. 12 was used as the corresponding equation Eq between the pulse transit time and the blood pressure in order to calculate (estimate) the blood pressure on the basis of the pulse transit time (PTT). However, the present invention is not limited to this. As the corresponding equation Eq between the pulse transit time and the blood pressure, there may be used an equation including a term of 1/DT and a term of DT in addition to the 1/DT², for example, as illustrated in Equation (Eq. 3) of FIG. 13 when the pulse transit time is represented as DT and the blood pressure is represented as EBP. In Equation (Eq. 3), each of α, β, γ and δ represents a known coefficient or constant.

Moreover, for example, as illustrated in Equation (Eq. 4) of FIG. 14, there may be used an equation including the term of 1/DT, a term of a cardiac cycle RR, and a term of a volume/pulse wave area ratio VR (for example, refer to Japanese Unexamined Patent Application Publication No. 2000-33078). In Equation (Eq. 4), each of α, β, γ and δ represents a known coefficient or constant. Note that, in this case, the CPU 100 calculates the cardiac cycle RR and the volume/pulse wave area ratio VR on the basis of the pulse wave signals PS1 and PS2.

Also, in the case of using Equation (Eq. 3) and Equation (Eq. 4) as the corresponding equation Eq between the pulse transit time and the blood pressure, the measurement accuracy for the blood pressure can be improved as in the case of using Equation (Eq. 2). Naturally, corresponding equation other than Equations (Eq. 2), (Eq. 3) and (Eq. 4) may be used.

In the above-mentioned embodiment, the first pulse wave sensor 40-1 and the second pulse wave sensor 40-2 detect, as a change of the impedance, the pulse wave of the artery (the radial artery 91) passing through the measurement site (the left wrist 90) (impedance system). However, the present invention is not limited to this. Each of the first and second pulse wave sensors may be provided with a light emitting element that applies light toward an artery passing through a corresponding portion of the measurement site and with a light receiving element that receives reflected light (or transmitted light) of the applied light, and may detect the pulse wave of the artery as a change of volume (photoelectric system). Moreover, each of the first and second pulse wave sensors may be provided with a piezoelectric sensor caused to abut against the measurement site, and may detect, as a change of electrical resistance, a pressure-derived distortion of the artery passing through the corresponding portion of the measurement site (piezoelectric system). Furthermore, each of the first and second pulse wave sensors may be provided with a transmission element that sends a radio wave (transmission wave) toward the artery passing through the corresponding portion of the measurement site and with a reception element that receives a reflected wave of the radio wave, and may detect a change of distance between the artery and the sensor, which is caused by the pulse wave of the artery, as a phase shift between the transmission wave and the reflected wave (radio wave application system).

Moreover, in the above-mentioned embodiment, the sphygmomanometer 1 is to be attached to the left wrist 90 as the measurement site. However, the present invention is not limited to this. The measurement site just needs to be a portion through which the artery passes, and may be an arm such as an upper arm other than the wrist, or may be a leg such as an ankle and a thigh.

Moreover, in the above-mentioned embodiment, the CPU 100 mounted on the sphygmomanometer 1 works as the search processing unit, the cross-correlation coefficient calculation unit, the measurement processing unit, and the first and second blood pressure calculation units, and executes the blood pressure measurement by the oscillometric method (operation flow of FIG. 6) and the blood pressure measurement (estimation) based on the PTT (operation flow of FIG. 8). However, the present invention is not limited to this. For example, a substantial computer device such as a smartphone, which is provided outside of the sphygmomanometer 1, may work as the search processing unit, the cross-correlation coefficient calculation unit, the measurement processing unit, and the first and second blood pressure calculation units, and via the network 900, may cause the sphygmomanometer 1 to execute the blood pressure measurement by the oscillometric method (operation flow of FIG. 6) and the blood pressure measurement (estimation) based on the PTT (operation flow of FIG. 8).

As is described above, a pulse wave measurement device of the present disclosure comprises:

a belt to be attached around a measurement site;

first and second pulse wave sensors which are mounted on the belt in a state of being spaced apart from each other in a width direction of the belt and detect pulse waves at portions of an artery passing through the measurement site, the portions individually facing the first and second pulse wave sensors;

a pressing member that is mounted on the belt and is capable of pressing the first and second pulse wave sensors against the measurement site while varying a pressing force;

a cross-correlation coefficient calculation unit that acquires first and second pulse wave signals which the first and second pulse wave sensors output respectively in a time series and calculates a cross-correlation coefficient between waveforms of the first and second pulse wave signals;

a search processing unit that varies and sets the pressing force generated by the pressing member and determines whether the cross-correlation coefficient which the cross-correlation coefficient calculation unit calculates exceeds a predetermined threshold value; and

a measurement processing unit that sets the pressing force generated by the pressing member to a value determined by the search processing unit, at which the cross-correlation coefficient exceeds the threshold value, and in a state of the pressing force's setting kept, acquires a time difference between the first and second pulse wave signals as a pulse transit time.

In this description, “measurement site” refers to a region through which an artery passes. For example, the measurement site may be an arm such as a wrist and an upper arm, or may be a leg such as an ankle and a thigh.

Moreover, “belt” refers to a member of band-like shape attached around the measurement site, regardless of its name. For example, the name of the belt may be “band”, “cuff” or the like in place of the belt.

Moreover, “width direction” of the belt corresponds to a longitudinal direction of the measurement site.

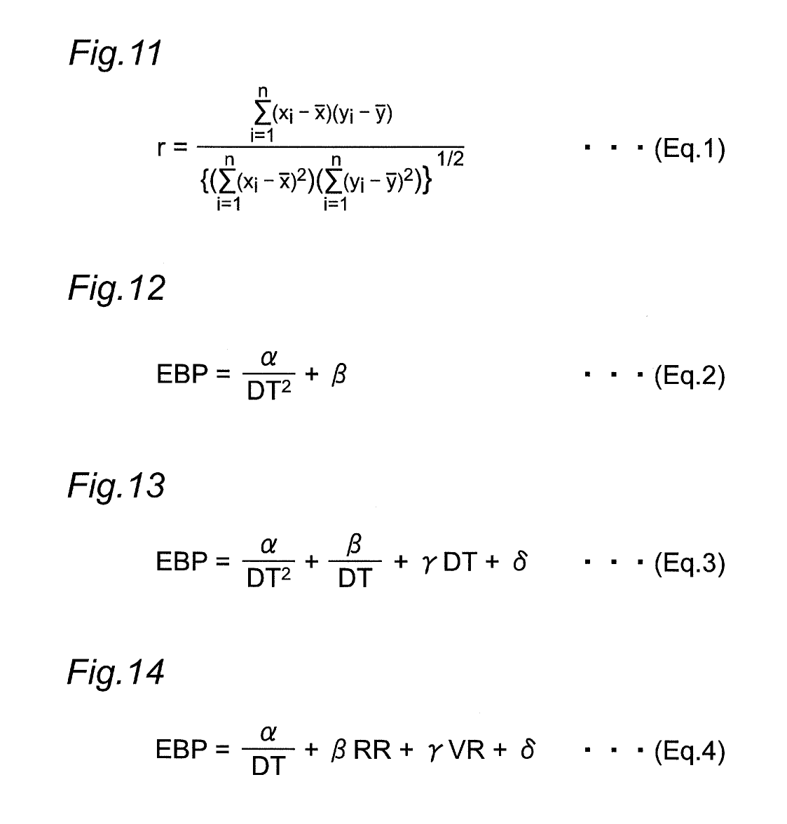

Furthermore, “cross-correlation coefficient” means a sample correlation coefficient (also referred to as Pearson product-moment correlation coefficient). For example, when data string {xᵢ} and a data string {yᵢ} which are composed of two sets of numeric values (where i=1, 2 . . . , n) are given, a cross-correlation coefficient r between the data string {xᵢ} and the data string {yᵢ} is defined by Equation (Eq. 1) illustrated in FIG. 11. Herein, x and y added with top bars in Equation (Eq. 1) represent average values of x and y, respectively.

In the pulse wave measurement device of this disclosure, the first and second pulse wave sensors are mounted on the belt in a state of being spaced from each other with respect to the width direction of the belt. In a state where the belt is attached around the measurement site, the pressing member presses the first and second pulse wave sensors against the measurement site, for example, by a certain pressing force. In this state, the first and second pulse wave sensors detect pulse waves at portions of an artery passing through the measurement site, the portions individually facing the first and second pulse wave sensors. The cross-correlation coefficient calculation unit acquires first and second pulse wave signals which the first and second pulse wave sensors output respectively in a time series, and calculates a cross-correlation coefficient between waveforms of the pulse wave signals. Herein, the search processing unit varies and sets the pressing force generated by the pressing member, and regarding the pressing force, determines whether the cross-correlation coefficient which the cross-correlation coefficient calculation unit calculates exceeds a predetermined threshold value. The measurement processing unit sets the pressing force generated by the pressing member to a value determined by the search processing unit, at which the cross-correlation coefficient exceeds the threshold value, and in a state of the pressing force's setting kept, acquires a time difference between the first and second pulse wave signals as a pulse transit time. In this way, the measurement accuracy for the pulse transit time can be improved.

In the pulse wave measurement device of one embodiment, the search processing unit gradually increases the pressing force generated by the pressing member from a time of starting an operation until the cross-correlation coefficient exceeds the threshold value, and the measurement processing unit sets the pressing force generated by the pressing member to a value at a point of time when the cross-correlation coefficient exceeds the threshold value, and acquires the pulse transit time.

To “gradually” increase the pressing force includes the case of continuously varying and increasing the pressing force and the case of increasing the pressing force step by step.

In the pulse wave measurement device of this embodiment, the pulse transit time can be acquired without unnecessarily increasing the pressing force for pressing the measurement site. In this way, a physical load on the user can be reduced.

In the pulse wave measurement device of one embodiment, the measurement processing unit sets the pressing force generated by the pressing member to a value at which the cross-correlation coefficient exhibits a maximum value, and acquires the pulse transit time.

In accordance with an experiment by the inventor of the present disclosure, it was found that, when the pressing forces against the measurement site at the first and second pulse wave sensors were gradually increased from zero, then the cross-correlation coefficient was gradually increased following the increase of the pressing forces, exhibited a maximum value, and then was gradually decreased. Accordingly, in the pulse wave measurement device of this embodiment, the measurement processing unit sets the pressing force generated by the pressing member to a value at which the cross-correlation coefficient exhibits the maximum value, and acquires the pulse transit time. In this way, the measurement accuracy for the pulse transit time can be further improved.

In the pulse wave measurement device of one embodiment, the first and second pulse wave sensors include respectively first and second pairs of detection electrodes disposed on an inner circumferential surface of the belt, and using the first and second pairs of detection electrodes, output, as the first and second pulse wave signals, signals which represent impedances of portions of the measurement site, the portions facing the first and second pairs of detection electrodes.

In this description, “signal which represent impedances” include, as well as signals which directly represent impedances, signals indirectly represent impedances, for example, such as drop voltages in the case where an alternating constant current flows through the measurement site.

In the pulse wave measurement device of this embodiment, the first and second pulse wave sensors include respectively first and second pairs of detection electrodes disposed on an inner circumferential surface of the belt, and using the first and second pairs of detection electrodes, output signals which represent impedances of portions of the measurement site, the portions facing the first and second pairs of detection electrodes, as the first and second pulse wave signals. The pairs of detection electrodes as described above can be composed to be flat, for example, by plate-shaped or sheet-shaped electrodes. Hence, in this pulse wave measurement device, the belt can be composed to be thin.

In the pulse wave measurement device of one embodiment, the measurement processing unit acquires as the pulse transit time, a time difference between a peak of the first pulse wave signal and a peak of the second pulse wave signal.

In another aspect, a blood pressure measurement device of the present disclosure comprises:

the above-described pulse wave measurement device; and

a first blood pressure calculation unit that calculates a blood pressure based on the pulse transit time, which is acquired by the measurement processing unit, using a predetermined corresponding equation between the pulse transit time and the blood pressure.

In the blood pressure measurement device of this embodiment, the pulse transit time is acquired with accuracy by (the measurement processing unit of) the pulse wave measurement device. Using a predetermined corresponding equation between the pulse transit time and the blood pressure, the first blood pressure calculation unit calculates (estimates) a blood pressure on the basis of the pulse transit time acquired by the measurement processing unit. Hence, the measurement accuracy for the blood pressure can be improved.

In the blood pressure measurement device of one embodiment,

the pressing member is a fluid bag provided along the belt,

a body provided integrally with the belt is provided, and

on the body, there are mounted:

the search processing unit, the measurement processing unit and the first blood pressure calculation unit; and

for blood pressure measurement by an oscillometric method, a pressure control unit that supplies the fluid bag with air and controls a pressure of the air, and a second blood pressure calculation unit that calculates the blood pressure based on the pressure of the air in the fluid bag.

In this description, the fact that the body is “integrally provided” with the belt may be that the belt and the body may be subjected, for example, to integral molding, or in place thereof, may be that the belt and the body are formed separately from each other and the body is integrally attached with the belt via an engagement member (for example, a hinge or the like).

In the pulse wave measurement device of this embodiment, the blood pressure measurement (estimation) based on the pulse transit time and the blood pressure measurement by the oscillometric method can be performed by an integrated device. Hence, convenience for the user is improved.

In another aspect, a pulse wave measurement method of the present disclosure is a pulse wave measurement method of measuring a pulse wave of a measurement site by including:

a belt to be attached around the measurement site;

first and second pulse wave sensors mounted on the belt in a state of being spaced apart from each other with in a width direction of the belt; and

a pressing member that is mounted on the belt and is capable of pressing the first and second pulse wave sensors against the measurement site while varying a pressing force, the pulse wave measurement method comprising:

detecting pulse waves at portions of an artery passing through the measurement site by the first and the second pulse wave sensors in a state where the belt is attached around the measurement site and the pressing member presses the first and second pulse wave sensors against the measurement site by a certain pressing force, the portions individually facing the first and second pulse wave sensors;

acquiring first and second pulse wave signals which the first and second pulse wave sensors output respectively in a time series and calculating a cross-correlation coefficient between waveforms of the pulse wave signals;

varying and setting the pressing force generated by the pressing member and determining whether the cross-correlation coefficient exceeds a predetermined threshold value regarding the pressing force; and

setting the pressing force generated by the pressing member to a value determined by the search processing unit, at which the cross-correlation coefficient exceeds the threshold value, and in a state of the pressing force's setting kept, acquiring a time difference between the first and second pulse wave signals as a pulse transit time.

In accordance with the pulse wave measurement method of this disclosure, the measurement accuracy for the pulse transit time can be improved.

As apparent from the above, in accordance with the pulse wave measurement device and pulse wave measurement method of this disclosure, the measurement accuracy for the pulse transit time can be improved.

Moreover, in accordance with the blood pressure measurement device of this disclosure, the measurement accuracy for the blood pressure can be improved.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A pulse wave measurement device comprising:
a belt to be attached around a measurement site;
first and second pulse wave sensors which are mounted on the belt in a state of being spaced apart from each other in a width direction of the belt and detect pulse waves at portions of an artery passing through the measurement site, the portions individually facing the first and second pulse wave sensors;
a pressing member that is mounted on the belt and is capable of pressing the first and second pulse wave sensors against the measurement site while varying a pressing force;
a cross-correlation coefficient calculation unit that acquires first and second pulse wave signals which the first and second pulse wave sensors output respectively in a time series and calculates a cross-correlation coefficient between waveforms of the first and second pulse wave signals;
a search processing unit that varies and sets the pressing force generated by the pressing member and determines whether the cross-correlation coefficient which the cross-correlation coefficient calculation unit calculates exceeds a predetermined threshold value; and
a measurement processing unit that sets the pressing force generated by the pressing member to a value determined by the search processing unit, at which the cross-correlation coefficient exceeds the threshold value, and in a state of the pressing force's setting kept, acquires a time difference between the first and second pulse wave signals as a pulse transit time.

2. The pulse wave measurement device according to claim 1, wherein
the search processing unit gradually increases the pressing force generated by the pressing member from a time of starting an operation until the cross-correlation coefficient exceeds the threshold value, and
the measurement processing unit sets the pressing force generated by the pressing member to a value at a point of time when the cross-correlation coefficient exceeds the threshold value, and acquires the pulse transit time.

3. The pulse wave measurement device according to claim 1, wherein
the measurement processing unit sets the pressing force generated by the pressing member to a value at which the cross-correlation coefficient exhibits a maximum value, and acquires the pulse transit time.

4. The pulse wave measurement device according to any one of claim 1, wherein the first and second pulse wave sensors include respectively first and second pairs of detection electrodes disposed on an inner circumferential surface of the belt, and using the first and second pairs of detection electrodes, output, as the first and second pulse wave signals, signals which represent impedances of portions of the measurement site, the portions facing the first and second pairs of detection electrodes.

5. A blood pressure measurement device comprising:
the pulse wave measurement device according to any one of claim 1; and
a first blood pressure calculation unit that calculates a blood pressure based on the pulse transit time, which is acquired by the measurement processing unit, using a predetermined corresponding equation between the pulse transit time and the blood pressure.

6. The blood pressure measurement device according to claim 5, wherein
the pressing member is a fluid bag provided along the belt,
a body provided integrally with the belt is provided, and
on the body, there are mounted:
the search processing unit, the measurement processing unit and the first blood pressure calculation unit; and
for blood pressure measurement by an oscillometric method, a pressure control unit that supplies the fluid bag with air and controls a pressure of the air, and a second blood pressure calculation unit that calculates the blood pressure based on the pressure of the air in the fluid bag.

7. A pulse wave measurement method of measuring a pulse wave of a measurement site by including:
a belt to be attached around the measurement site;
first and second pulse wave sensors mounted on the belt in a state of being spaced apart from each other with in a width direction of the belt; and
a pressing member that is mounted on the belt and is capable of pressing the first and second pulse wave sensors against the measurement site while varying a pressing force, the pulse wave measurement method comprising:
detecting pulse waves at portions of an artery passing through the measurement site by the first and the second pulse wave sensors in a state where the belt is attached around the measurement site and the pressing member presses the first and second pulse wave sensors against the measurement site by a certain pressing force, the portions individually facing the first and second pulse wave sensors;
acquiring first and second pulse wave signals which the first and second pulse wave sensors output respectively in a time series and calculating a cross-correlation coefficient between waveforms of the pulse wave signals;
varying and setting the pressing force generated by the pressing member and determining whether the cross-correlation coefficient exceeds a predetermined threshold value regarding the pressing force; and
setting the pressing force generated by the pressing member to a value determined by the search processing unit, at which the cross-correlation coefficient exceeds the threshold value, and in a state of the pressing force's setting kept, acquiring a time difference between the first and second pulse wave signals as a pulse transit time.

8. The pulse wave measurement device according to any one of claim 1, wherein
the measurement processing unit acquires as the pulse transit time, a time difference between a peak of the first pulse wave signal and a peak of the second pulse wave signal.
